(19) United States
(12) Patent Application Publication
(10) Pub. No.: US 2017/0304636 A1
(43) Pub. Date: Oct. 26, 2017

(54) SYSTEM FOR INCREASED TEMPORAL FLEXIBILITY IN A MEDICAL DEVICE

(71) Applicant: Boston Scientific Neuromodulation Corporation, Valencia, CA (US)

(72) Inventors: G. Karl Steinke, Valencia, CA (US); Michael A. Moffitt, Saugus, CA (US); Stephen Carcieri, Los Angeles, CA (US)

(21) Appl. No.: 15/492,095

(22) Filed: Apr. 20, 2017

Related U.S. Application Data
(60) Provisional application No. 62/325,374, filed on Apr. 20, 2016.

(51) Int. Cl. A61N 1/372; A61N 1/36

(52) U.S. Cl. CPC A61N 1/37247; A61N 1/36178; A61N 1/37241

(57) ABSTRACT

In various embodiments, the present system allows for custom definition of waveforms and waveform segments for neurotherapy. Such custom definition is achieved by using a waveform composer with a user interface that makes it possible for the user to perform the custom definition of potentially very complex patterns of neurostimulation waveforms by creating and editing graphical representations of relatively simple individual building blocks for each of the patterns.

### PRIORITY CLAIM

This patent application claims the benefit of priority, under 35 U.S.C. Section 119 to U.S. Provisional Patent Application Ser. No. 62/325,374, entitled “SYSTEM FOR INCREASED TEMPORAL FLEXIBILITY IN A MEDICAL DEVICE,” filed on Apr. 20, 2016 to Steinke et al, which is hereby incorporated by reference herein in its entirety.

### TECHNICAL FIELD

Embodiments pertain to medical devices. Some embodiments relate to implantable medical devices which deliver electrical stimulation. Some embodiments relate to implantable neurostimulation devices.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

The delivery of the neurostimulation energy is controlled using stimulation parameters that specify spatial (where to stimulate), temporal (when to stimulate), and informational patterns of energy directing the nervous system to respond as desired) aspects of a pattern of neurostimulation pulses. Many current neurostimulation systems are programmed to deliver periodic pulses with one or a few uniform stimulations continuously or in bursts. However, the human nervous systems use neural signals having much more sophisticated patterns to communicate various types of information, including sensations of pain, pressure, temperature, etc. The nervous system may interpret an artificial stimulation with a simple pattern of stimuli as an unnatural phenomenon, and respond with an unintended and undesirable sensation and/or movement, or ignore the signal over time, resulting in loss of efficiency or efficacy. For example, some neurostimulation therapies are known to cause paresthesia and/or sensation of vibration of non-targeted tissues or organs.

### SUMMARY

The following are non-limiting examples.

Example 1 is a system comprising: a composer device comprising: a processor; a memory communicatively coupled to the processor and comprising instructions, which when executed by the processor, cause the processor to perform operations comprising: providing a composer graphical user interface (GUI), the composer GUI providing a waveform editor, the waveform editor permitting a user to create a waveform for providing neurostimulation therapy; building a composition comprising information on the waveform, the composition in a first format; and sending the composition to a programmer, the programmer programming an implantable medical device with stimulation parameters in a second format different than the first format, based on the composition.

In Example 2, the subject matter of Example 1 optionally includes a programmer device comprising: a second processor; a second memory communicatively coupled to the second processor and comprising instructions, which when executed by the processor, cause the processor to perform operations comprising: receiving the composition; connecting to the implantable medical device; translating the waveform of the composition into a plurality of stimulation parameters; and uploading the plurality of stimulation parameters to the implantable medical device.

In Example 3, the subject matter of Example 2 optionally includes wherein the programmer operations comprise: providing a programmer GUI, the programmer GUI displaying information on the waveform; determining that a first parameter of the waveform is marked in the composition as read only; and in response, preventing editing of the first parameter by a user of the programmer device.

In Example 4, the subject matter of Example 3 optionally includes wherein the programmer operations comprise: connecting to the implantable medical device; creating a test waveform from the waveform, the test waveform a waveform that is shorter in length and comprising at least one waveform segment with a maximum stimulation of the waveform.

In Example 5, the subject matter of any one or more of Examples 1-4 optionally include, wherein the programmer is executing on the processor, and wherein the operations of sending the composition to the programmer comprises one or more of: sending the composition using interprocess communication, or storing the composition in a data storage device in a location accessible by the programmer.

In Example 6, the subject matter of any one or more of Examples 1-5 optionally include, wherein a first portion of the composition is marked in the composition as read only.

In Example 7, the subject matter of any one or more of Examples 1-6 optionally include, wherein a first portion of the composition is locked to prevent a first parameter of the waveform from being modified by the programmer.

In Example 8, the subject matter of any one or more of Examples 1-7 optionally include, comprising uploading the composition to a waveform library.

In Example 9, the subject matter of any one or more of Examples 1-8 optionally include, wherein the composer GUI allows the user to create a waveform segment family comprising a plurality of waveform segments in the waveform matching criteria defined by the user, and wherein the operations further comprises changing a characteristic of all waveform segments in the waveform segment family responsive to a user input.

Example 10 is a method comprising: using one or more computer processors: providing a composer graphical user interface (GUI), the composer GUI providing a waveform editor, the waveform editor permitting a user to create a waveform for providing neurostimulation therapy; building a composition comprising information on the waveform, the composition in a first format; and sending the composition to a programmer, the programmer programming an implantable medical device with stimulation parameters in a second format different than the first format, based on the composition.

In Example 11, the subject matter of Example 10 optionally includes wherein the waveform comprises at least one sequence comprising one or more trains, the one or more trains comprising one or more bursts, the one or more bursts comprising one or more pulses.

In Example 12, the subject matter of any one or more of Examples 10-11 optionally include wherein the programmer is executing on the one or more computer processors, and wherein sending the composition to the programmer comprises one or more of: sending the composition using interprocess communication, or storing the composition in a data storage device in a location accessible by the programmer.

In Example 13, the subject matter of any one or more of Examples 10-12 optionally include wherein the programmer is executing on a separate programming device and wherein sending the composition to the programmer comprises sending the composition over a network.

In Example 14, the subject matter of any one or more of Examples 10-13 optionally include wherein a first parameter of the waveform is marked in the composition as read only.

Example 15 is a machine readable medium comprising instructions, which when performed by a machine, causes the machine to perform the operations to perform the methods of Examples 10-14.

Example 16 is a method comprising: using one or more computer processors: providing a composer graphical user interface (GUI), the composer GUI providing a waveform editor, the waveform editor permitting a user to create a waveform for providing neurostimulation therapy; building a composition comprising information on the waveform, the composition in a first format; and sending the composition to a programmer, the programmer programming an implantable medical device with stimulation parameters in a second format different than the first format, based on the composition.

In Example 17, the subject matter of Example 16 optionally includes wherein the waveform comprises at least one sequence comprising one or more trains, the one or more trains comprising one or more bursts, the one or more bursts comprising one or more pulses.

In Example 18, the subject matter of any one or more of Examples 16-17 optionally include wherein the programmer is executing on the one or more computer processors, and wherein sending the composition to the programmer comprises one or more of: sending the composition using interprocess communication, or storing the composition in a data storage device in a location accessible by the programmer.

In Example 19, the subject matter of any one or more of Examples 16-18 optionally include wherein the programmer is executing on a separate programming device and wherein sending the composition to the programmer comprises sending the composition over a network.

In Example 20, the subject matter of any one or more of Examples 16-19 optionally include wherein a first parameter of the waveform is marked in the composition as read only.

In Example 21, the subject matter of any one or more of Examples 16-20 optionally include wherein a first portion of the composition is locked to prevent a first parameter of the waveform to prevent modification by the programmer.

In Example 22, the subject matter of any one or more of Examples 16-21 optionally include uploading the composition to a waveform library.

In Example 23, the subject matter of any one or more of Examples 16-22 optionally include wherein the composer GUI allows the user to create a waveform segment family comprising a plurality of waveform segments in the waveform matching criteria defined by the user, and wherein the method further comprises changing a characteristic of all waveform segments in the pulse family responsive to a user input.

Example 24 is a system comprising: a composer device comprising: a processor; a memory communicatively coupled to the processor and comprising instructions, which when executed by the processor, cause the processor to perform operations comprising: providing a composer graphical user interface (GUI), the composer GUI providing a waveform editor, the waveform editor permitting a user to create a waveform for providing neurostimulation therapy; building a composition comprising information on the waveform, the composition in a first format; and sending the composition to a programmer, the programmer programming an implantable medical device with stimulation parameters in a second format different than the first format, based on the composition.

In Example 25, the subject matter of Example 24 optionally includes a programmer device comprising: a second processor; a second memory communicatively coupled to the second processor and comprising instructions, which when executed by the processor, cause the processor to perform operations comprising: receiving the composition; connecting to the implantable medical device; translating the waveform of the composition into a plurality of stimulation parameters; and uploading the plurality of stimulation parameters to the implantable medical device.

In Example 26, the subject matter of Example 25 optionally includes wherein the programmer operations comprise: providing a programmer GUI, the programmer GUI displaying information on the waveform; determining that a first parameter of the waveform is marked in the composition as read only; and in response, preventing editing of the first parameter by a user of the programmer device.

In Example 27, the subject matter of Example 26 optionally includes wherein the programmer operations comprise: connecting to the implantable medical device; creating a test waveform from the waveform, the test waveform a waveform that is shorter in length and comprising at least one waveform segment with a maximum stimulation of the waveform.

In Example 28, the subject matter of any one or more of Examples 24-27 optionally include wherein the programmer is executing on the processor, and wherein the operations of sending the composition to the programmer comprises one or more of: sending the composition using interprocess communication, or storing the composition in a data storage device in a location accessible by the programmer.

In Example 29, the subject matter of any one or more of Examples 24-28 optionally include wherein a first portion of the composition is marked in the composition as read only.

In Example 30, the subject matter of any one or more of Examples 24-29 optionally include wherein a first portion of the composition is locked to prevent a first parameter of the waveform from being modified by the programmer.

In Example 31, the subject matter of any one or more of Examples 24-30 optionally include uploading the composition to a waveform library.

In Example 32, the subject matter of any one or more of Examples 24-31 optionally include wherein the composer GUI allows the user to create a waveform segment family comprising a plurality of waveform segments in the waveform matching criteria defined by the user, and wherein the operations further comprises changing a characteristic of all waveform segments in the waveform segment family responsive to a user input.

Example 33 is a machine-readable medium, comprising instructions, which when performed by a machine, causes the machine to perform operations comprising: providing a composer graphical user interface (GUI), the composer GUI providing a waveform editor, the waveform editor permitting a user to create a waveform for providing neurostimulation therapy; building a composition comprising information on the waveform, the composition in a first format; and sending the composition to a programmer, the programmer programming an implantable medical device with stimulation parameters in a second format different than the first format, based on the composition.

In Example 34, the subject matter of Example 33 optionally includes wherein the waveform comprises one or more sequences comprising one or more trains, the one or more trains comprising one or more bursts, the one or more bursts comprising one or more pulses.

In Example 35, the subject matter of any one or more of Examples 33-34 optionally include wherein the operations comprise running the programmer, and wherein the operations of sending the composition to the programmer comprises one or more of: sending the composition using interprocess communication, or storing the composition in a data storage device in a location accessible by the programmer.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a block diagram of a neurostimulation system according to some examples of the present disclosure.

FIG. 2 illustrates a block diagram of a neurostimulation system according to some examples of the present disclosure.

FIG. 3 illustrates a block diagram of a neurostimulation system according to some examples of the present disclosure.

FIG. 4 illustrates a block diagram of a neurostimulation system according to some examples of the present disclosure.

FIG. 5 illustrates a block diagram of a stimulation device and a lead system according to some examples of the present disclosure.

FIG. 6 illustrates a block diagram of a programming device according to some examples of the present disclosure.

FIG. 7 illustrates a block diagram of a programming device according to some examples of the present disclosure.

FIG. 8 shows a block diagram of a composer device according to some examples of the present disclosure.

FIG. 9 shows a block diagram of a waveform library according to some examples of the present disclosure.

FIG. 10 illustrates an implantable neurostimulation system and portions of an environment in which system may be used according to some examples of the present disclosure.

FIG. 11 shows a block diagram of an implantable stimulator and one or more electrodes according to some examples of the present disclosure.

FIG. 12 illustrates a composition flow from a waveform library or composer to the programmer and implantable device according to some examples of the present disclosure.

FIG. 13 shows a flowchart of a method of a composer device according to some examples of the present disclosure.

FIG. 14 shows a flowchart of a method of a programmer according to some examples of the present disclosure.

FIG. 15 shows a flowchart of a method of an implantable medical device according to some examples of the present disclosure.

FIG. 16 shows a flowchart of a method of an implantable medical device according to some examples of the present disclosure.

FIG. 17 shows a flowchart of a method of a programmer or composer device accessing a waveform library according to some examples of the present disclosure.

FIG. 18 shows a flowchart of a method of testing a program prior to deployment on the implantable medical device according to some examples of the present disclosure.

FIG. 19 is a block diagram illustrating an example of a machine upon which one or more embodiments may be implemented.

### DETAILED DESCRIPTION

Recent research has shown that the efficacy and efficiency of certain neurostimulation therapies can be improved, and their side-effects can be reduced, by using neurostimulation signals that emulate neural signals observed in the human body. While modern electronics can accommodate the need for generating such sophisticated pulse patterns, the capability of a neurostimulation system depends on its post-manufacturing programmability to a great extent. For example, a sophisticated pulse pattern may only benefit a patient when it is customized for that patient, and stimulation patterns predetermined at the time of manufacturing may substantially limit the potential for the customization. Such customization may be performed at least in part by a user such as a physician or other caregiver with the patient in a clinical setting.

Advancements in neuroscience and neurostimulation research have led to a demand for using complex and/or individually optimized patterns of neurostimulation energy for various types of therapies. For example, a greater demand for control of neurostimulation in a temporal domain. The capability of a neurostimulation system in treating various types of disorders will be limited by the programmability of such patterns of neurostimulation pulses.

In various embodiments, the present system allows for custom definition of one or more waveforms using a waveform composer. A waveform may include one or more discrete pulses or other discrete waveforms where the waveform is separated from adjacent waveforms, or may include continuous waveforms. A waveform may include waveform segments that function as building blocks for the waveform. The waveform segments may be concatenated, forming a chain of waveform segments that are used to build the overall waveform that may be delivered over a stimulation channel. Also, waveform segments may be used to build other waveform segments, and those waveform segments may be used to build other waveform segments in a hierarchical structure with two or more levels of waveform segments. Thus, some waveform segments may be simple, elementary building blocks of the waveform, and other waveform segments may be more complex building blocks of the waveform. A waveform segment may include a digital and/or analog signal, and may include a variety of waveform shapes. A digital signal may be a square waveform or other digital waveform with stepped up or stepped down amplitudes. An analog waveform may have slopes that are not stepped.

Each waveform segment may have a waveform shape. The waveform shape may include a number of waveform features such as, by way of example and not limitation, peaks, valleys, zero-crossings, etc. for continuous waveform segments or such as, by way of example and not limitation, pulse amplitude, pulse width, pulse-to-pulse spacing for discrete waveform segments. Waveform features may form a pattern. The pattern of a given waveform segment may be perceived as a regular or repeatable pattern or may be perceived as an inconsistent pattern that does not appear to repeat in a given waveform segment. Multiple waveform segments may be used to provide an overall waveform that can be streamed over a stimulation channel that may appear to be a regularly repeating pattern of waveform features or may appear to be a complex, inconsistent pattern of waveform features. Furthermore, some embodiments may be capable of providing waveforms on two or more stimulation channels. For example, some embodiments may be configured to implement four timing channels where each timing channel can provide its own waveform and timing for the waveform.

Waveform segments may include a primary pulse feature and may further comprise pre-pulse feature(s) and/or post-pulse feature(s). By way of example and not limitation, pre-pulse and post-pulse feature(s) may allow a primary pulse to have an intended effect at a lower energy and/or may diminish or eliminate undesired effects of the primary pulse. Waveform segments may include other feature(s), such as an interphase feature to control timing between the phases of a biphasic pulse or charge recovery features to promote charge balancing to protect tissue and electrodes. One or more waveforms may be sent over a stimulation channel to selected, active electrodes. Multiple stimulation channels may be delivered simultaneously. Multiple waveforms may be delivered over one or more stimulation channels and combined to form a different waveform (e.g., additive or subtractive).

Custom definition of waveforms or their segments may be achieved in various embodiments by using a waveform composer with a user interface that enables the user to perform the custom definition of potentially very complex waveforms by creating and editing graphical representations of relatively simple individual building blocks (e.g., elementary waveform segments) for each of the waveforms. Waveforms in some examples may include a defined hierarchy of waveform segments such as but not limited to pulses, bursts of pulses, trains of bursts, and sequences of pulses, bursts, and trains. In some examples, this hierarchy is not present and waveforms may have any combination of segments. The composer may be located on the programming device (integrated into the programming software, or as a separate software application), or may be located on a different device.

In various embodiments, the present system provides for patterns of neurostimulation energy not limited to programs predefined at the time of manufacturing, thereby accommodating need for customization of neurostimulation as well as need for new types of neurostimulation waveforms that may, for example, result from future research in neurostimulation. This may also facilitate design of a general-purpose neurostimulation device that can be configured by a user for delivering specific types of neurostimulation therapies by programming the device using the user interface.

In various embodiments, the composer allows for creating and editing waveforms for neurostimulation starting with templates or presets to enable valuable time savings in defining complex waveforms and waveform segments. In various embodiments, the user interface provides for complete editorial control as well as simplified, guided, and template-based editorial options. The composer allows for editing waveform parameters. Waveform parameters refer to any aspect of the waveform including shape, composition, and the like.

In various embodiments, the present system may be implemented using a combination of hardware and software designed to provide users such as researchers, physicians or other caregivers, or neurostimulation device makers with ability to create custom waveforms in an effort to increase therapeutic efficacy and/or patient satisfaction for neurostimulation therapies, including but not being limited to SCS and DBS therapies. While neurostimulation is specifically discussed as an example, the present subject matter may apply to any therapy that employs stimulation waveforms of electrical or other form of energy.

The composer in some examples is integrated with a programmer device which communicates with the implantable neurostimulation device to transfer the waveform or waveform building blocks in the form of a set of stimulation parameters (e.g., a program) to the device. In these examples, the composer functionality may be provided as part of the neurostimulation configuration application on the programming device which allows for programming the neurostimulation device. In other examples, the composer device may be a different device that provides a user interface and which communicates waveforms to the programmer device which may allow editing of one or more aspects of the waveform. The programmer device may then communicate these defined waveforms to the neurostimulation device. The neurostimulation device may then utilize these parameters to deliver neurostimulation therapy.

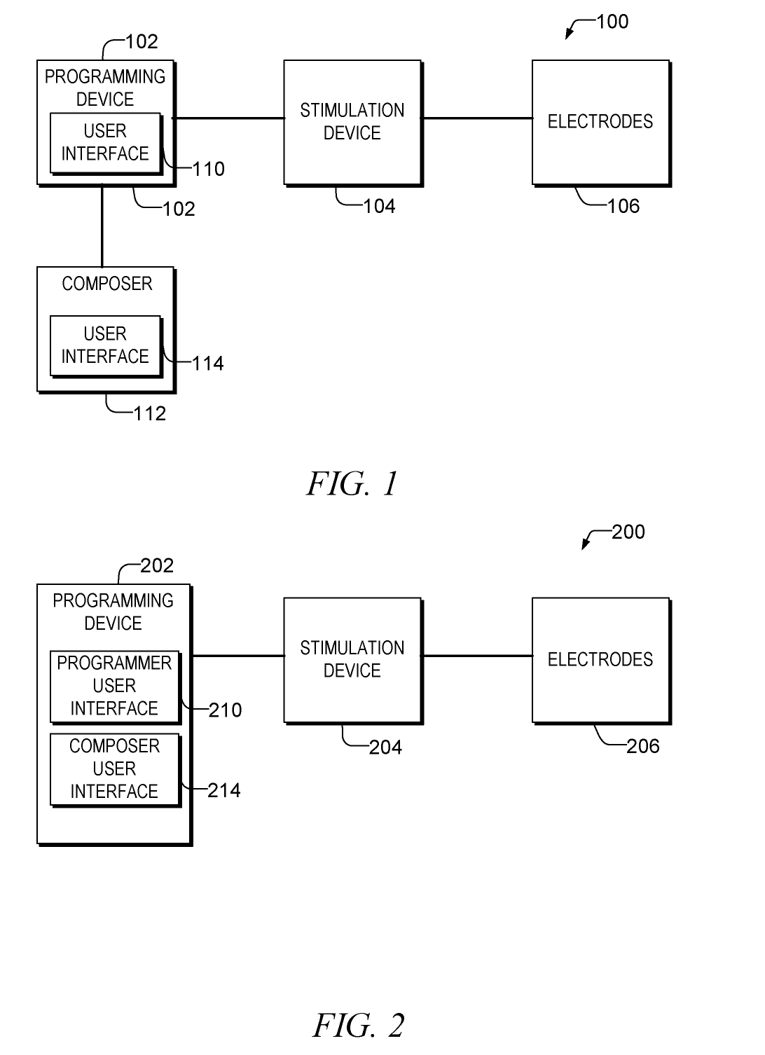

FIG. 1 illustrates a block diagram of a neurostimulation system 100 according to some examples of the present disclosure. Neurostimulation system 100 includes electrodes 106, a stimulation device 104, a programming device 102, and in some examples a composer device 112. Electrodes 106 are configured to be placed on or near one or more targets in a patient. Stimulation device 104 is configured to be electrically connected to electrodes 106 and deliver neurostimulation energy, such as in the form of electrical pulses, to the one or more targets though electrodes 106. The delivery of the neurostimulation is controlled by using a plurality of stimulation parameters, such as described by a program of stimulation parameters, specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using neurostimulation system 100. Programming device 102 provides the user with accessibility to the user-programmable parameters. In various embodiments, programming device 102 is configured to be communicatively coupled to stimulation device via a wired or wireless link. In various embodiments, programming device 102 includes a user interface 110 that allows the user to set and/or adjust values of the user-programmable parameters by creating and/or editing graphical representations of various programs.

Composer device 112 may include a user interface 114 which provides a user with the ability to create and edit one or more waveforms or waveforms building blocks. Such waveforms may include, for example, the waveform to be used to deliver therapy to the patient as well as waveform segments that can be used to build waveforms. Examples of such waveform segments includes, but is not limited to, pulses, bursts (each burst including a group of one or more pulses), trains (each including a group of bursts), and sequences (each including a group of the pulses, bursts, and trains), as further discussed below. In various embodiments, programming device 102 allows the user to edit existing waveforms or waveform building blocks, create new waveforms or waveform building blocks, import waveforms or waveform building blocks created by other users, and/or export waveform building blocks to be used by other users. The user may also be allowed to define an electrode selection specific to each waveform building block.

In some examples, the user interface 110 may allow a user to view, select, and download one or more waveforms or waveform building blocks developed on composer device 112 (e.g., stored on composer device 112 or in a network library). Once downloaded, user interface 110 of programming device 102 may allow the user to edit the waveform or waveform building blocks. For example, the user may copy, edit, or move the waveform building blocks to create new waveforms or edit existing waveforms. In some examples, only a subset of the stimulation parameters are editable in the user interface 110 of the programming device 102. This may allow the medical device manufacturer to define waveforms or waveform segments and allow for a simplified amount of changes at the programming device 102 Waveforms, waveform building blocks, or parameters within the waveform building blocks or waveforms may have associated access protections, such as read-only, read/write, or locked (e.g., neither readable or writeable).

In some examples, certain waveform segments may not be modified, whereas the programming device 102 may modify any aspect of other waveform segments. In other examples, certain waveform segments may not be modified, whereas the programming device 102 may modify only certain aspects of other waveform settings. In yet other examples, the waveform may have one or more safety features that allows programming device 102 to edit waveform segments, but restricts the programming device 102 from editing the waveform in a way that exceeds one or more predetermined safety parameters. An example safety parameter may include a total energy delivered over a predetermined period of time. These safety parameters may be specified in the composition describing the waveform or waveform segment.

FIG. 2 illustrates a block diagram of a neurostimulation system 200 according to some examples of the present disclosure. Neurostimulation system 200 includes electrodes 206, a stimulation device 204, and a programming device 202. In the example of FIG. 2, rather than having a separate composer device as was the case in FIG. 1, the composer user interface 214 is part of the programming device, as is the programmer user interface 210. In some examples, the programmer user interface 210 and the composer user interface 214 are integrated such that the functionality of the composer user interface 214 and the programmer user interface 210 are available from the same user interface. The programmer user interface 210 and the composer user interface 214 may be provided by the same software application executing on the programming device 202 or a different software application executing on the programming device.

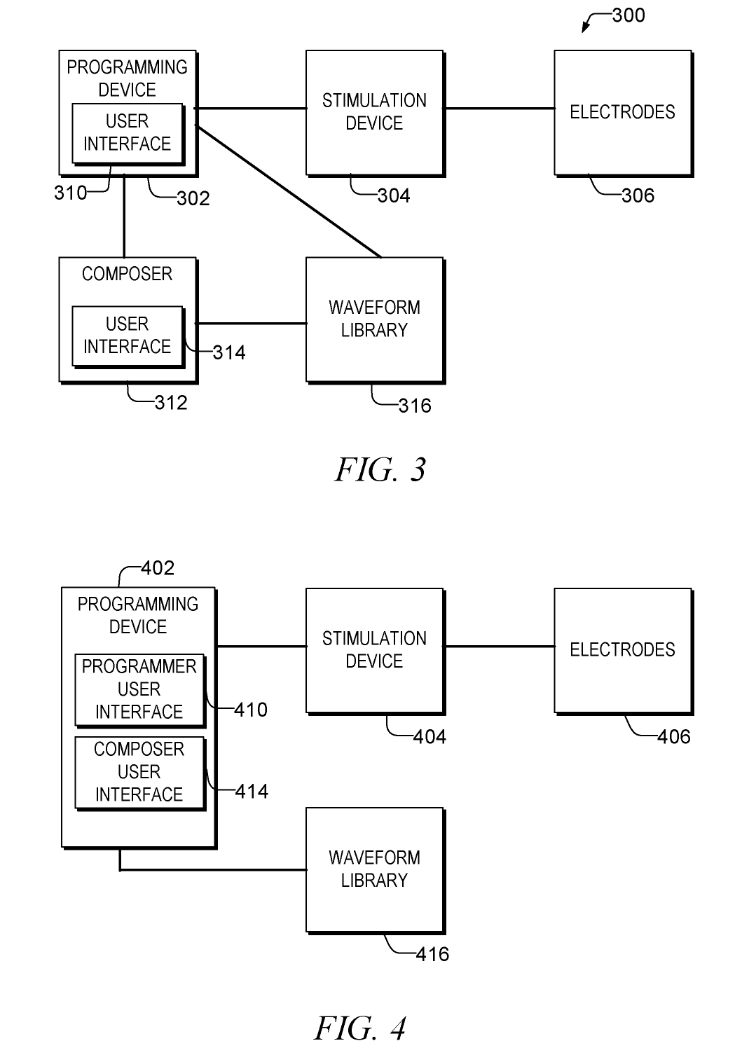

FIG. 3 illustrates a block diagram of a neurostimulation system 300 according to some examples of the present disclosure. Neurostimulation system 300 includes electrodes 306, a stimulation device 304, a programming device 302 (with user interface 310), a composer device 312 (with a composer user interface 314), and a waveform library 316. Electrodes 306, stimulation device 304, programming device 302, user interface 310, composer device 312, composer user interface 314 have the same functions as their corresponding components from FIG. 1, with the exception of an interface between the programming device 302 and composer 312 to the waveform library 316. The waveform library 316 may be a network-accessible library of waveforms as well as a network-accessible library of waveform segments. These waveforms or waveform segments may be downloadable by a user of the composer device 312 or programming device 302 and programmed onto the stimulation device 304 for delivery through electrodes 306.

FIG. 4 illustrates a block diagram of a neurostimulation system 400 according to some examples of the present disclosure. Neurostimulation system 400 includes electrodes 406, a stimulation device 404, a programming device 402, and a stimulation library 416. The programmer user interface 410 and composer user interface 414 are both provided by one or more software applications executing on the programming device 402. Electrodes 406, stimulation device 404, programming device 402, user interface 410, composer user interface 414 have the same functions as their corresponding components from FIG. 2, with the exception of an interface between the programming device 402 to the waveform library 416. The waveform library may be a network-accessible library of waveforms and waveform segments. These waveforms and waveform segments may be downloadable by a user of the programming device 402 and programmed onto the stimulation device 404 for delivery through electrodes 406.

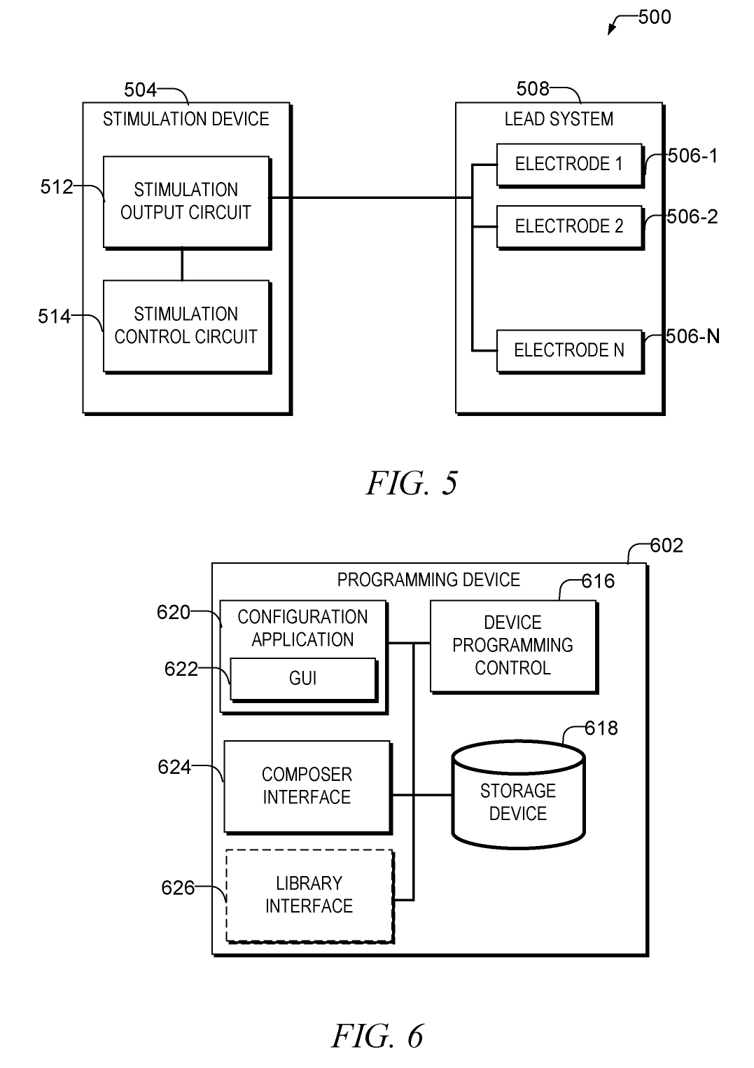

FIG. 5 illustrates a block diagram of a stimulation device 504 and a lead system 508, such as may be implemented in neurostimulation system 100 according to some examples of the present disclosure. Stimulation device 504 represents an embodiment of stimulation device 104, 204, 304, and 404 and includes a stimulation output circuit 512 and a stimulation control circuit 514. Stimulation output circuit 512 produces and delivers neurostimulation pulses. Stimulation control circuit 514 controls the delivery of the neurostimulation pulses using the plurality of stimulation parameters, which specifies a pattern or patterns of the neurostimulation pulses. Lead system 508 includes one or more leads each configured to be electrically connected to stimulation device 504 and a plurality of electrodes 506 distributed in the one or more leads. The plurality of electrodes 506 includes electrode 506-1, electrode 506-2, . . . electrode 506-N, each a single electrically conductive contact providing for an electrical interface between stimulation output circuit 512 and tissue of the patient, where N>2. The waveforms are each delivered from stimulation output circuit 512 through a set of one or more electrodes selected from electrodes 506.

In various embodiments, the number of leads and the number of electrodes on each lead depend on, for example, the distribution of target(s) of the neurostimulation and the need for controlling the distribution of electric field at each target. In one embodiment, lead system 508 includes 2 leads each having 8 electrodes.

FIG. 6 illustrates a block diagram of a programming device 602, such as may be implemented in neurostimulation system 100 according to some examples of the present disclosure. Programming device 602 represents an embodiment of programming device 102 and 302, and includes a storage device 618, a device programming control circuit 616, a configuration application 620 (which provides Graphical User Interface 622), a composer interface 624, and an optional library interface 626. Storage device 618 may store a plurality of waveforms or waveform segments. Device programming control circuit 616 communicates with the implantable medical device. For example, device programming control circuit communicates one or more stimulation parameters to the implantable medical device that implements one or more waveforms or waveform segments, and reads one or more configuration settings and sensor values from the implantable medical device. Device programming control circuit 616 works with the configuration application 620 to determine stimulation parameters to transmit to the implantable medical device and provides information received from the implantable medical device to the configuration application 620. Device programming control circuit 616 may implement one or more communication protocols used to communicate with the implantable medical device.

Configuration application 620 generates the plurality of stimulation parameters that controls the delivery of the neurostimulation pulses according to the waveform selected by a user through the GUI 622. GUI 622 represents an embodiment of user interface 110, 310. GUI 622 may utilize one or more waveforms, or waveform segments received from the composer or the library.

Composer interface 624 may implement one or more communication protocols to communicate with the composer to allow users of the programming device 602 to browse, select, and download waveforms or waveform building blocks available at the composer. Example protocols include HyperText Transfer Protocol (HTTP), File Transfer Protocol (FTP), User Datagram Protocol (UDP), Transport Control Protocol (TCP), Internet Protocol (IP), and the like. For example, the composer interface 624 may receive one or more user interface descriptors from the composer. Example user interface descriptors include HyperText Markup Language (HTML), JavaScript, Content Style Sheets (CSS), eXtensible Markup Language (XML), and other documents. These documents may be renderable in the programming device, such as through the use of an Internet Browser or the GUI 622 to provide a selection of waveforms or waveform segments available at the composer. Users may browse the available waveforms including descriptions of the waveforms and meta data about the waveforms (e.g., creator, date created, information about the program, and the like). Users may then download one or more waveforms or waveform segments and import those waveforms or waveform segments into the GUI 622 for editing, review, and/or programming to the implantable device.

Optional library interface 626 may implement one or more communication protocols to communicate with a waveform library to allow users of the programming device 602 to browse, select, and download waveforms or waveform segments from the waveform library. For example, the user may license or purchase a program (e.g., by paying royalties or other fees) from the library. In some examples, a basic set of sequences may be downloaded initially from the factory. As noted, sequences may also be uploaded to the library. Example protocols include HyperText Transfer Protocol (HTTP), File Transfer Protocol (FTP), User Datagram Protocol (UDP), Transport Control Protocol (TCP), Internet Protocol (IP), and the like. For example, the library interface 626 may receive one or more user interface descriptors from the waveform library. Example user interface descriptors include HyperText Markup Language (HTML), JavaScript, Content Style Sheets (CSS), eXtensible Markup Language (XML), and other documents. These documents may be renderable in the programming device, such as through the use of an Internet Browser or the GUI 622 to provide a user interface of the waveform library. Users may browse the available waveforms and waveform segments including descriptions of the waveforms and waveform segments and meta data about them (e.g., creator, date created, information about the program, and the like). Users may then download one or more waveforms and waveform segments and import them into the GUI 622 for editing, review, and/or programming to the implantable device.

In various embodiments, GUI 622 allows for editing and creating various features of waveforms and their segments, including segments such as but not limited to pulses, trains, and bursts. For example GUI 622 may allow users to create their own waveforms or waveform building blocks (segments). In some examples, the GUI 622 may not allow users to create their own waveforms, and instead, will allow users the ability to choose one or more predefined waveforms or waveform segments, or programs available from the composer or the library and in some examples make limited changes to the waveforms or the waveform segments.

In some examples, GUI 622 may allow users to edit one or more waveforms or waveform segments received from a composer device or from a waveform library through composer interface 624 and library interface 626 respectively. In some examples, the GUI 622 is not allowed to modify waveforms or waveform segments downloaded from the composer device or waveform library. In yet other examples, the GUI 622 may modify only certain portions of the waveform or waveform segments. The portions of the waveform or waveform segments that the GUI 622 may not modify may be coded into the GUI 622 at manufacture time. In other examples, the portions of the waveform or waveform segments the GUI 622 may not modify may be specified in a data structure received from the library or from the composer that describes the waveform or waveform segments. The structure may be called a composition. A composition is a file or other data structure which stores information describing one or more waveforms or waveform segments and in some examples stores information on which waveforms to deliver to which one or more electrodes (e.g. active electrode(s)) and the timing of this delivery. In this way, the portion of a program that the GUI 622 may modify may differ from waveform to waveform. For example, some waveforms may allow users of GUI 622 to modify the pulse width only, different waveforms may allow users of the GUI 622 to modify both the pulse width and amplitude, and the like. In other examples, the composition may specify every parameter is editable, while other composition may specify that no parameters are editable. The GUI 622 reads the permissible modifications from information in the composition.

In some examples, the impermissible modifications are specified in a field of the composition, but in other examples, portions of the composition that are not editable by the GUI 622 may be locked (e.g., encrypted), or otherwise made unreadable to the programmer by the composer or library. For example, the composer and/or library may possess a first key in a cryptographic key pair and use that key to encrypt the portions of the waveform that are not editable by the GUI 622. The implantable medical device may possess the second key in the pair and thus may be able to decrypt those portions. The programming device 602 may not possess one of the keys and thus will not be able to decrypt or edit these portions.

In one embodiment, programming device 602 may take input from a touchscreen. In various embodiments, GUI 622 may output to any type of presentation device, such as interactive or non-interactive screens, and take input from any type of user input devices that allow the user to edit the waveforms or waveform segments, such as touchscreen, keyboard, keypad, touchpad, trackball, joystick, and mouse. In various embodiments, circuits of programming device 602, including its various embodiments discussed in this document, may be implemented using a combination of hardware and software. In some examples, the circuits, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

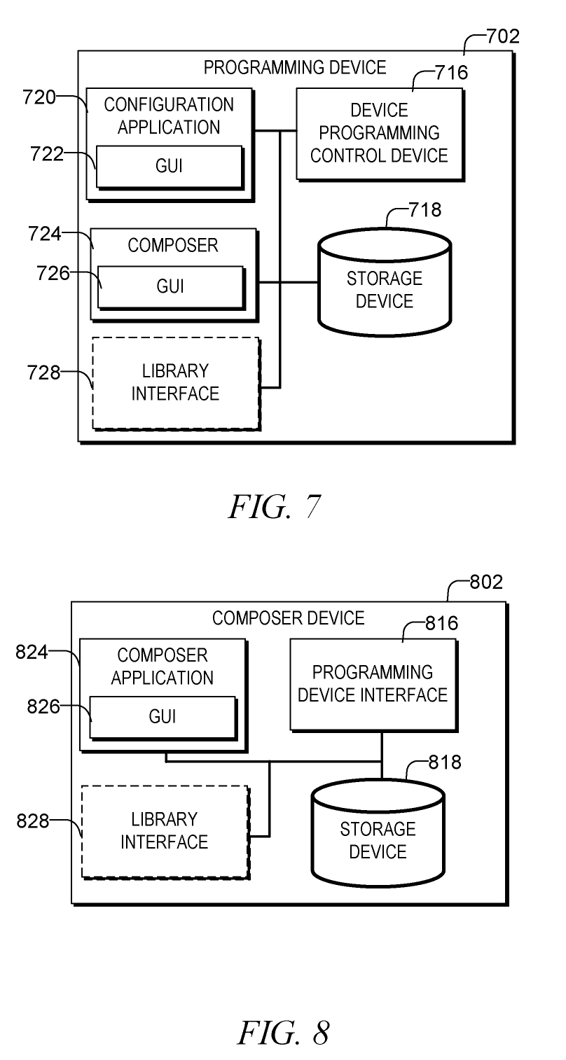

FIG. 7 illustrates a block diagram of a programming device 702, such as may be implemented in neurostimulation system 200,400 according to some examples of the present disclosure. Programming device 702 represents an embodiment of programming device 202 and 402 and includes a storage device 718, a device programming control circuit 716, a configuration application 720 implementing a GUI 722, a composer application 724 implementing a GUI 726, and an optional library interface 728. Storage device 718 may store one or more waveforms and waveform segments. Device programming control circuit 716 communicates with the implantable medical device in the same way described for device programming control circuit 616. Configuration application 720 and GUI 722 allow users to build, store in storage device 718, load from storage device 718, and/or edit waveforms and waveform segments and otherwise program the implantable device as is described for configuration application 620 and GUI 622.

Composer application 724 provides GUI 726 that allows the user to create and manage waveforms and waveform segments, including creating and importing waveforms and waveform segments (e.g., such as pulses, trains, bursts, and sequences of pulses, sequences of trains, and sequences of bursts) to be added to the waveforms and waveform segments stored in storage device 718, exporting waveforms and waveform segments selected from waveforms and waveform segments stored in storage device 718, and editing each of the waveforms and waveform segments. In various embodiments, GUI 726 allows for graphical editing of each of the waveforms and waveform segments. In various embodiments, composer application 724 allows the user to compose the waveform to be delivered to the patent using stimulation device 104 using waveform segments such as pulses, bursts each including a group of the pulses, trains each including a group of the bursts, and/or sequences each including a group of the pulses, bursts, and trains. In various embodiments, composer application 724 allows the user to create each waveform or waveform segment using one or more waveforms or waveform segments stored in storage device 718 as templates. For example, the user may copy, edit, and move building blocks around to create waveforms and their segments. These blocks may be repeated for a determined duration. The duration of the blocks may also be edited. In some examples, the user may create a waveform with one or more empty parameters that may be filled in prior to delivery of the waveform by one or more of the components of the system. In various embodiments, composer application 724 allows each newly created waveform or waveform segment to be saved as additional waveform or waveform segment stored in storage device 718. Composer application 724 may create a separate GUI 726, but in other examples, the GUI 726 may be integrated with GUI 722. Likewise the functionality of composer application 724 may be provided by a separate application or may be integrated into the configuration application 720.

In some examples, the composer application 724 may summarize the characteristics of a waveform or waveform segment in a synopsis. The synopsis may comprise descriptive statistics about the waveform or the waveform segments. For example, statistics about individual pulses within the waveform or waveform segment such as one or more of: amplitude, pulse width, inter-pulse intervals, pulse shape, number of phases per pulse, and the like. The composer application 724 may then allow users to search for portions of waveforms or waveform segments based upon these characteristics. For example, the composer application 724 may return all waveforms or waveform segments (e.g., pulses) matching these search criteria. The search results may comprise a waveform segment “family.” In some examples, all the waveform segments of this family may be directly edited such that changes to these parameters apply to all waveform segments in this family in the waveform. In some examples, once a change is made, a new program is saved to storage device 718, but in other examples, edits are made and saved to the waveform being modified.

Optional library interface 728 may implement one or more communication protocols to communicate with a waveform library to allow users of the programming device 702 to browse, select, and download waveforms and waveform segments in the waveform library as is described for library interface 628. Users may browse the available waveforms and waveform segments including descriptions of the waveforms and waveform segments and meta data about the waveforms and waveform segments (e.g., creator, date created, information about the waveform and waveform segments, and the like). Users may then download one or more waveforms or waveform segments and import those waveforms and waveform segments into the GUI 722 or GUI 726. Example protocols include HyperText Transfer Protocol (HTTP), File Transfer Protocol (FTP), User Datagram Protocol (UDP), Transport Control Protocol (TCP), Internet Protocol (IP), and the like.

In one embodiment, GUIs 722, 726 may take input from a touchscreen. In various embodiments, GUIs 722, 726 may output to any type of presentation device, such as interactive or non-interactive screens, and take input from any type of user input devices that allow the user to edit the waveforms and waveform segments or building blocks, such as touchscreen, keyboard, keypad, touchpad, trackball, joystick, and mouse. In various embodiments, circuits of programming device 702, including its various embodiments discussed in this document, may be implemented using a combination of hardware and software. In some examples, the circuits, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 8 shows a block diagram of a composer device 802 according to some examples of the present disclosure. Programming device interface 816 may implement one or more communication protocols to communicate with a programming device to allow users of the programming device to browse, select, and download waveforms and waveform segments in the storage device 818. For example, the programming device interface 816 may create and send one or more user interface descriptors (e.g., as part of a web-based application). Example user interface descriptors include HyperText Markup Language (HTML), JavaScript, Content Style Sheets (CSS), eXtensible Markup Language (XML), and other documents. For example, programming device interface 816 may comprise a web-server functionality. These documents may be renderable in the programming device, such as through the use of an Internet Browser to provide a user interface. Example protocols include HyperText Transfer Protocol (HTTP), File Transfer Protocol (FTP), User Datagram Protocol (UDP), Transport Control Protocol (TCP), Internet Protocol (IP), and the like. Users may browse the available waveforms and waveform segments including descriptions of the waveforms and waveform segments and meta data about the waveforms and waveform segments (e.g., creator, date created, information about the waveforms and waveform segments, and the like). Users may then download one or more waveforms and waveform segments and import those waveforms and waveform segments.

Composer application 824 may provide GUI 826. Composer application 824 and GUI 826 allows for the functionality as described in composer application 724 and GUI 726. In various embodiments, composer application 824 provides GUI 826 that allows the user to create, edit, and importing waveforms and waveform segments (e.g., pulses, trains, bursts, and sequences of pulses, sequences of trains, and sequences of bursts).

Optional library interface 828 may implement one or more communication protocols to communicate with a waveform library to allow users of the composer device 802 to browse, select, and download waveforms and waveform segments in the waveform library. Example protocols include HyperText Transfer Protocol (HTTP), File Transfer Protocol (FTP), User Datagram Protocol (UDP), Transport Control Protocol (TCP), Internet Protocol (IP), and the like. For example, the library interface 828 may receive one or more user interface descriptors from the waveform library. Example user interface descriptors include HyperText Markup Language (HTML), JavaScript, Content Style Sheets (CSS), eXtensible Markup Language (XML), and other documents. These documents may be renderable in the composer device 802, such as through the use of an Internet Browser or the GUI 826 to provide a user interface of the waveform library. Users may browse the available waveforms and waveform segments including descriptions of the waveforms and waveform segments and meta data about the waveforms and waveform segments (e.g., creator, date created, information about the waveforms and waveform segments, and the like). Users may then download one or more waveforms and waveform segments and import those waveforms and waveform segments into the GUI 826 for modification or review.

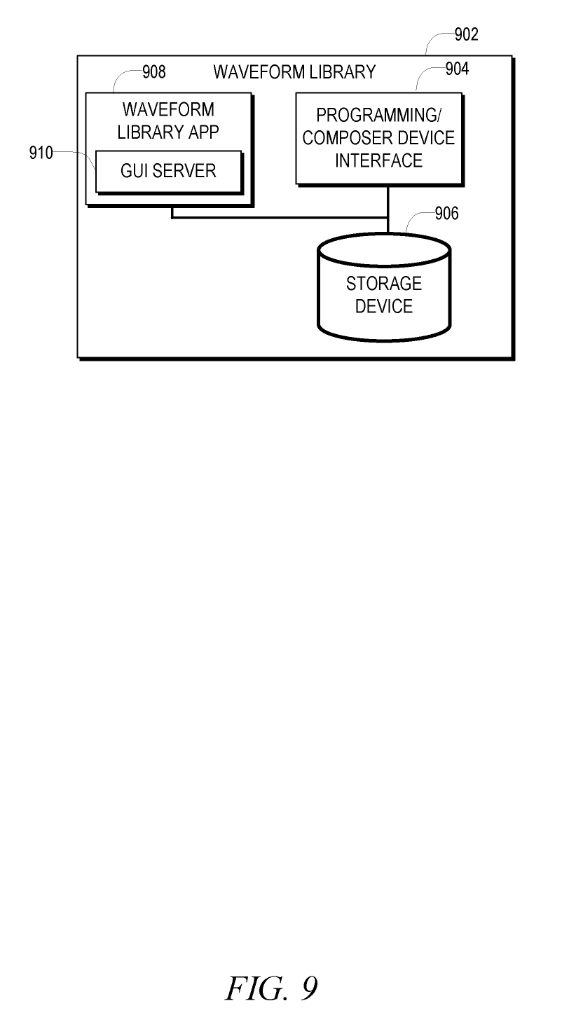

FIG. 9 shows a block diagram of a waveform library 902 according to some examples of the present disclosure. Programming/Composer device interface 904 provides for implementing one or more communication protocols to communicate with the programmer or the composer device to allow those devices to browse, select, and download waveforms and waveform segments stored in storage device 906. Programming/Composer device interface 904 also provides for implementing one or more communication protocols to allow the programmer or composer device to upload one or more waveforms and waveform segments for storage in storage device 906 to allow other programmers or other composer devices to download those waveforms and waveform segments. Example protocols include HyperText Transfer Protocol (HTTP), File Transfer Protocol (FTP), User Datagram Protocol (UDP), Transport Control Protocol (TCP), Internet Protocol (IP), and the like. Waveform library application 908 and GUI server 910 may work with programming/composer device interface to provide one or more user interface descriptors. Example user interface descriptors include HyperText Markup Language (HTML), JavaScript, Content Style Sheets (CSS), eXtensible Markup Language (XML), and other documents. For example, programming/composer device interface 904 may comprise a web-server functionality. These documents may be renderable in the programming or composer device, such as through the use of an Internet Browser, GUI, or other application, to provide a user interface. Users may browse the available waveforms and waveform segments including descriptions of the waveforms and waveform segments and meta data about the waveforms and waveform segments (e.g., creator, date created, information about the waveforms and waveform segments, and the like). Users may then download one or more waveforms or waveform segments. Users may also upload their own waveforms and waveform segments through these interfaces. Waveform library 902 may sell waveforms or waveform segments for purchase or license and may accept payment prior to sending a waveform or a waveform segment.

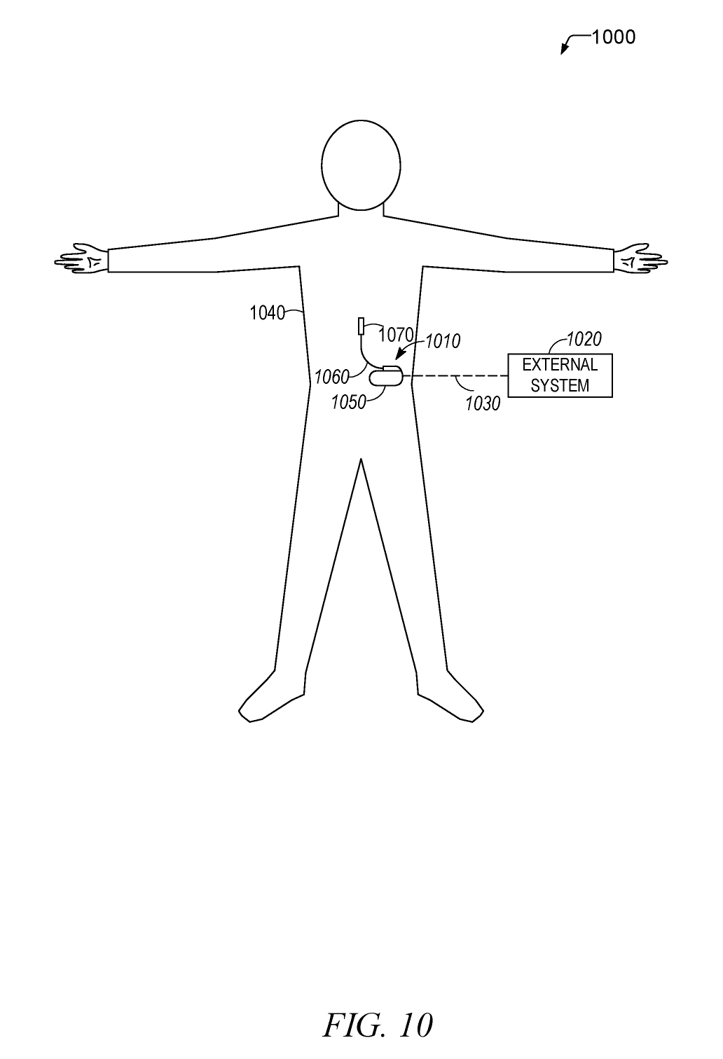

FIG. 10 illustrates an implantable neurostimulation system 1000 and portions of an environment in which system 1000 may be used according to some examples of the present disclosure. System 1000 includes an implantable system 1010, an external system 1020, and a telemetry link 1030 providing for wireless communication between implantable system 1010 and external system 1020. Implantable system 1010 is illustrated in FIG. 10 as being implanted in the patient's body 1040.

Implantable system 1010 includes an implantable stimulator (also referred to as an implantable pulse generator, or IPG) 1050, a lead system 1060, and electrodes 1070, which represent an embodiment of stimulation device 504; lead system 508, and electrodes 506, respectively. External system 1020 represents an embodiment of programming device 602, 702. In various embodiments, external system 1020 includes one or more external (non-implantable) devices each allowing the user and/or the patient to communicate with implantable system 1010. In some embodiments, external system 1020 includes a programming device intended for the user to initialize and adjust settings for implantable stimulator 1050 and a remote control device intended for use by the patient. For example, the remote control device may allow the patient to turn implantable stimulator 1050 on and off and/or adjust certain patient-programmable parameters of the plurality of stimulation parameters.

The sizes and shapes of the elements of implantable system 1010 and their location in body 1040 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regardless of stimulation targets in the patient's body and whether the stimulation device is implantable.

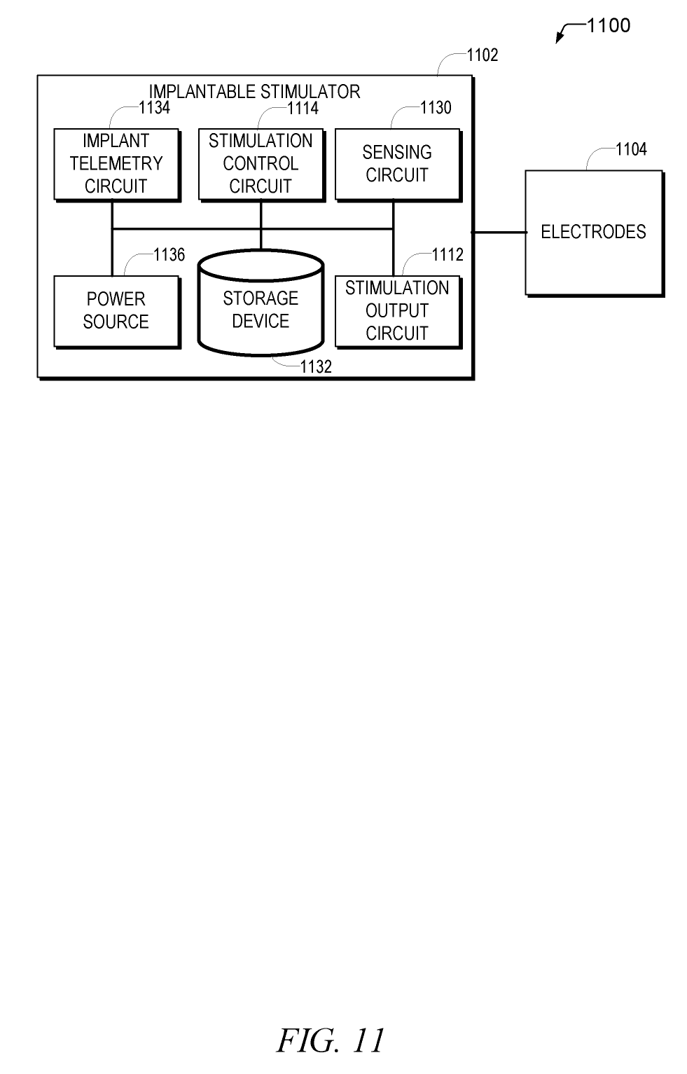

FIG. 11 shows a block diagram of an implantable stimulator 1102 and one or more electrodes 1104 of an implantable neurostimulation system, such as implantable system 1100, according to some examples of the present disclosure. Implantable stimulator 1102 may include a sensing circuit 1130 that is optional to implement a sensing capability, stimulation output circuit 1112, a stimulation control circuit 1114, an implant storage device 1132, an implant telemetry circuit 1134, and a power source 1136. Sensing circuit 1130, when included, senses one or more physiological signals for purposes of patient monitoring and/or feedback control of the neurostimulation. Examples of the one or more physiological signals includes neural and other signals each indicative of a condition of the patient that is treated by the neurostimulation and/or a response of the patient to the delivery of the neurostimulation. Stimulation output circuit 1112 is electrically connected to electrodes 1104 through leads, and delivers each of the neurostimulation pulses through a set of electrodes selected from electrodes 1104. Stimulation control circuit 1114 represents an embodiment of stimulation control circuit 1114 and controls the delivery of the neurostimulation pulses using the plurality of stimulation parameters specifying the pattern of the neurostimulation pulses. In one embodiment, stimulation control circuit 1114 controls the delivery of the neurostimulation pulses using the one or more sensed physiological signals. Implant telemetry circuit 1134 provides implantable stimulator 1102 with wireless communication with another device such as a device of an external system, including receiving values of the plurality of stimulation parameters from the external system. Implant storage device 1132 stores values of the plurality of stimulation parameters. Power source 1136 provides implantable stimulator 1102 with energy for its operation. In one embodiment, power source 1136 includes a battery. In one embodiment, power source 1136 includes a rechargeable battery and a battery charging circuit for charging the rechargeable battery. Implant telemetry circuit 1134 may also function as a power receiver that receives power transmitted from an external system through an inductive couple.

In various embodiments, sensing circuit 1130 (if included), stimulation output circuit 1112, stimulation control circuit 1114, implant telemetry circuit 1134, implant storage device 1132, and power source 1136 are encapsulated in a hermetically sealed implantable housing. In various embodiments, lead(s) are implanted such that electrodes 1104 are places on and/or around one or more targets to which the neurostimulation pulses are to be delivered, while implantable stimulator 1102 is subcutaneously implanted and connected to lead(s) at the time of implantation.

In some examples, the implantable stimulator 1102 may have various fault control, recognition, and handling procedures. For example, the stimulation control circuit 1114 may detect waveform corruption. For example, the stimulation parameters (representing the waveform) stored in storage device 1132 may have a signature (e.g., a checksum, hash, or other unique value calculable from the data of the stimulation parameters) associated with it stored in storage device 1132. Upon accessing the stimulation parameters either in the storage device 1132 or in an operating memory (e.g., Random Access Memory—not shown for clarity), the stimulation control circuit 1114 may calculate the signature and compare it to the signature that was stored in storage device 1132. If the calculated signature does not match the stored signature, the implantable stimulator 1102 may declare the stimulation parameters corrupt and take one or more defined actions.

Example actions include using a backup copy of the stimulation parameters, using a second set of backup stimulation parameters that were either manufacturing defaults, or are downloaded to the implantable stimulator 1102 from the programmer, or the like. In another example, the backup stimulation parameters may be computed by the implantable stimulator 1102. For example, the implantable stimulator may utilize a pre-programmed generic waveform. In some examples, the implantable stimulator may notify a user, for example, by communicating with an external remote control. In other examples, the implantable stimulator may notify a user when the implantable stimulator is next in communication with the programming device.

Implantable stimulator 1102 may also detect internal faults or other circumstances that require resetting or suspending stimulation. For example, if the implantable stimulator 1102 resets due to a software error, the presence of a strong magnetic field, or the like. For example, if the system resets, stimulation may be interrupted. In some examples, upon recovering, the stimulator has a number of options for resuming stimulation.

In one example, the stimulation resumes from the beginning of the waveform or from the beginning of a waveform segment (e.g., a pulse train). In another example, when stimulation is stopped, the waveform halts in place and when stimulation is resumed, the waveform resumes in the same place it left off. In another example, a clock tracks how long stimulation has been off and when stimulation is turned back on, the waveform resumes in the place where it was scheduled to be at if the stimulation hadn't been turned off. In yet another example, a clock tracks how long stimulation has been off and the time that stimulation has been off is used to make a decision as to whether to (a) start from the beginning of the waveform or the beginning of a waveform segment (b) resume the waveform in the place stimulation was halted, or (c) to resume where the waveform is scheduled to be at the current time. For example, the system may use two time thresholds, a first threshold, and a second threshold, where the second is greater than the first. If the time that stimulation is off exceeds neither threshold, the implantable stimulator 1102 resumes the waveform in the place stimulation was halted. If the time exceeds the first threshold but not the second, the implantable stimulator 1102 resumes stimulation in the place where the waveform is scheduled to be at the current time. If the time exceeds both the first and second thresholds, the implantable stimulator 1102 resumes from the beginning of the waveform or a waveform segment (depending on the configuration). The user may select which of the various options to use, or the system may be preprogrammed with a predetermined option. In yet other examples, each composition may specify one of the aforementioned actions. The aforementioned examples, may be independently programmable by stimulation channels or areas, such that synchronization of phases in the pulse trains from different stimulation channels or areas may be preserved if only one stimulation channel or area is turned off and then on again.

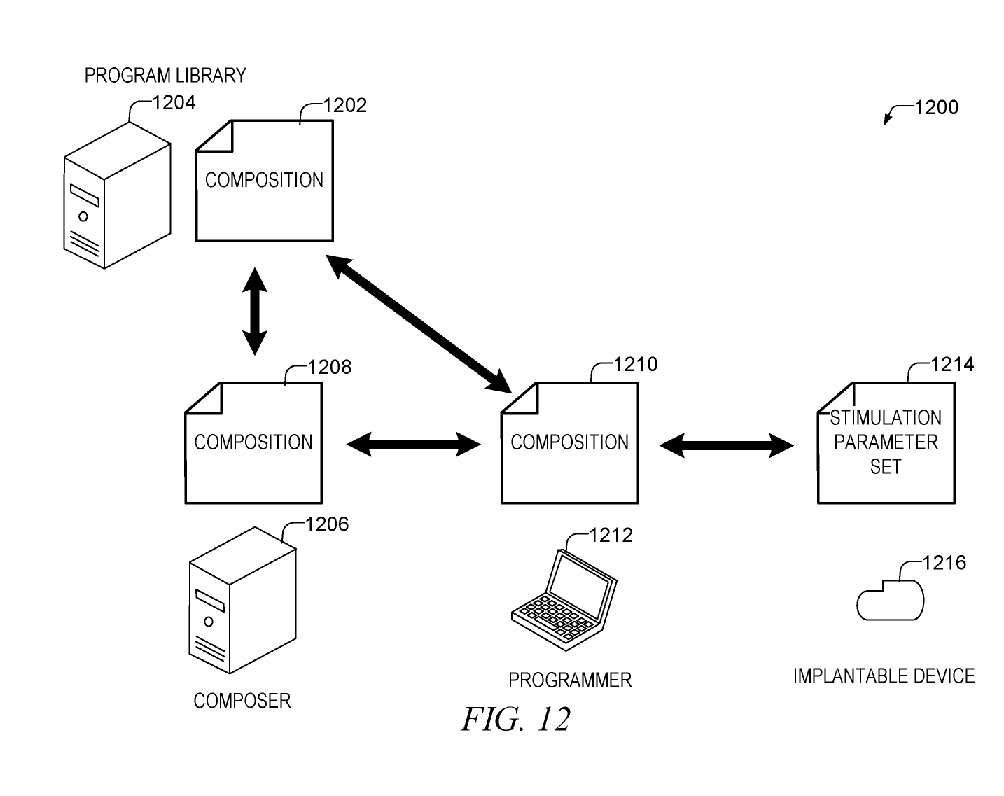

FIG. 12 illustrates a composition flow from a waveform library 1204 or composer 1206 to the programmer 1212 and implantable device 1216 according to some examples of the present disclosure. As already noted, a composition may refer to a file or data structure describing one or more waveforms or waveform segments which is then translated into a stimulator-interpretable set of parameters that define the stimulation (e.g., similar to how high level code is translated into machine instructions). The waveform library may store one or more compositions, such as composition 1202. Composer 1206 may download composition 1202 and may modify it, or otherwise change the format from a format stored in the waveform library 1204, to a format used by composer 1206. Thus, composition 1208 may be the same format and may describe the same program as composition 1202 or may be different. Likewise, programmer 1212 may receive composition 1202 or composition 1208 from waveform library 1204 or composer 1206, respectively. The format of the composition 1210 may be the same or different than composition 1202 and 1208. Programmer 1212 may utilize composition 1210 to generate stimulation parameter set 1214 (e.g., a “program”) to program the implantable device 1216. In some examples the stimulation parameters 1214 may be the same format as the composition files 1202, 1208, or 1210, but in other examples, the stimulation parameter set 1214 may be a different format. In some examples, the implantable device 1216 may reconstruct the program from the stimulation parameter set 1214. A composition may be created by a user of the composer 1206, but in other examples may be created based upon one or more programmings by the user at the programmer 1212.

In some examples, the programmer 1212 or composer 1206 may be able to reconstruct a particular composition 1210 or part of the composition 1210 from the stimulation parameter set 1214. For example, if the programmer 1212 was not the same programmer that programmed the stimulation parameter set 1214, or otherwise had no memory of the composition 1210, the programmer 1212 may read the stimulation parameter set 1214 from the memory of the implantable device 1216. The stimulation parameter set 1214 may allow for a full or partial reconstruction of the program to produce a composition 1210.

In some examples, as the composition file makes its way from the origin (e.g., composer or waveform library) to the implantable device 1216, the composition file may be stripped of non-essential information by waveform library 1204, composer 1206, programmer 1212, or implantable device 1216. Non-essential information is data that is not essential to producing the stimulation parameter set 1214 that specifies the program to the implantable device 1216. Example non-essential information is meta data about who created the program, when the program was created, statistics about the program, and other such information. This non-essential information may not be reconstructable by programmer 1212 or composer 1206 from only the stimulation parameter set 1214.

In some examples, waveform library 1204, composer 1206 and programmer 1210 may include one or more protections in composition files 1202, 1208, 1210. These protections prevent modification of one or more aspects of one or more of the waveforms or waveform segments described in the composition file. For example, the composer 1206 may specify one or more aspects of the waveform or waveform segments that are not modifiable by the programmer 1212. This may be done by designating certain portions of the composition file that describe these aspects as non-modifiable. In these examples, programmer 1212 may enforce the read-only nature of these portions, by not allowing a user of programmer 1212 to edit these portions. In other examples, composer 1206 may enforce this through encrypting portions of the composition 1208 to prevent editing. In these examples, the implantable device 1216 obtains or is manufactured with a corresponding cryptographic key to unlock these sections such that implantable device can read these parameters, but programmer 1212 is not given this key.

As noted, in some examples, the programmer 1212 is not able to make subsequent adjustments to any parameters of the waveform or waveform segments. In yet other examples, the programmer 1212 is able to adjust some or all of the parameters of the waveforms or waveform segments. These adjustments may be performed using basic or advanced user interfaces. These adjustments may be limited to one or more parameters of the waveform or waveform segment. For example, only amplitude control may be made available through the programmer 1212, and amplitude control may be enabled on a patient's remote control. To ensure patient safety, various boundaries may be programmed into the composition such as minimum and maximum amplitude adjustments. These boundaries may be per waveform, per segment, or per waveform building block. That is, the boundaries may vary for each pulse in the program, for each burst, for each train, and the like. In some examples, pulse width control is also available on the programmer 1212. This pulse width control may be further parameterized, such that adjusting a single pulse width setting will adjust all pulse widths in the composition, or all pulse widths in a particular building block of the composition. Pulse width control may be in terms of % change. The programmer 1212, may automatically impose limits on allowed changes due to other rules, e.g. in order to maintain charge balance, or to follow safety rules, or to ensure that the settings may be programmed, e.g. that no pulse width is longer than those which can be supported by the periods of the programmed frequencies.

A remote control provided to the patient that interfaces with the implantable device may adjust certain stimulation parameters, e.g. amplitude. In some embodiments, the programmer or composer may enable patient control of certain parameters through a remote control device, e.g. amplitude, pulse width, and the like. The programmer or composer may set limits of those parameters, e.g. min, max, and the methods of changing those parameters, e.g. stepsize, the manner that pulsewdiths are changed when an ‘increase’ or ‘decrease’ command is issued by the patient remote control, and the like. For example, the composer or programmer may identify in the program which pulses have adjustable pulse widths. In some examples, when all pulses in a composition have an identical pulse width, or some other predetermined criteria, pulse width control may be optionally enabled. In some examples, a “Frequency” control is used to compress or expand the time between pulse-to-pulse initiation, but the pulse properties are otherwise preserved. In some examples, when all pulses are delivered with a common frequency, frequency control may optionally be enabled. In some examples, when all pulses are delivered with a singular and common amplitude, amplitude control may be enabled.

Additionally, the composer or programmer may specify which parameters are adjusted by the pulse width and amplitude controls of the remote control of the patient. For example, the composer may “overload” the pulse width and amplitude controls such that they adjust different parameters of the waveform or waveform segment than pulse width or amplitude to offer additional patient options using a remote control with limited buttons or interfaces. In some examples, the composer might identify methods to ‘increase’ or ‘decrease’ stimulation, such that either or both amplitude or pulse width or frequency or other parameters or combinations of parameters are altered.

In some examples, the composition file may be a human readable and interpretable format. For example, an eXtensible Markup Language (XML) format, a Java Script Notation (JSON) format, or a custom plain text format. In yet other examples, the composition file may not be human readable or interpretable. In some examples, the composition file, or portions of the composition file may be encrypted to prevent tampering from unauthorized users. The format of composition 1202, 1208, 1210 and stimulation parameter set 1214 may be the same formats as each other or may be different formats from each other. Thus composition 1202 and 1208 may be human readable, but 1210 and 1214 may not.

While FIG. 12 illustrates a situation in which the programmer 1212 is a separate device from composer 1206 and waveform library 1204, as noted in other examples, these devices may be the same devices. The discussion of FIG. 12 still applies to this configuration. As noted, in some examples, the composer 1206 is integrated within the programmer 1212 in the configuration application, and available as an additional user interface screen. In another example, the composer 1206 is separate from the programmer 1212, and the workflow is separated as well, such that the composer may be used rarely, by a subset of programmers who are skilled in the art of creating compositions. In these embodiments, the composer may be a software application, for use on select systems (e.g., manufacturer controlled systems) or uncontrolled systems (user systems). For example, computing devices running WINDOWS® or APPLE OS® operating systems. Example computing devices include a personal computer, a desktop, a laptop, a tablet, a smartphone, and the like. While in some examples, the composer application or configuration application is a dedicated application installed locally, in other embodiments, the composer or configuration application may be a web-based application. For example, the composer device or programmer device may utilize a web browser to download a user interface of the composer or programmer. Also, FIG. 12 shows an example where the composition is translated to a stimulation parameter set by the programmer 1212, however, in other examples, other components shown may perform the translation. For example, the composer 1206, the program library 1204. In other examples, the composition 1202 may be translated by the implantable device 1216. For example, the implantable device 1216 may be configured to read the composition 1202 and translate that to stimulation. For example, the programmer 1212 may instruct the IPG to enter a “waveform programming mode” and send commands and instructions which are then converted to a stimulation parameter set 1214 by the implantable device 1216.

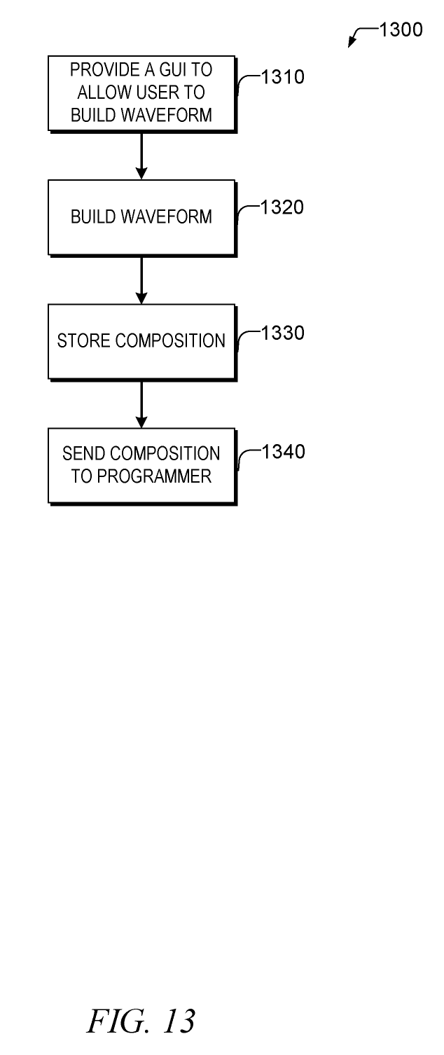

Turning now to FIG. 13 a flowchart of a method 1300 of a composer is shown according to some examples of the present disclosure. At operation 1310, the composer (either the same or different device than the device executing the configuration application and programmer GUI) provides a graphical user interface (GUI) to allow the user to build one or more waveforms or waveform segments. In some examples, this includes a user interface that allows the user to set and/or adjust user-programmable parameters of waveforms or waveform segments by creating and/or editing graphical representations of waveforms or waveform segments. Such waveforms or waveform segments may include, for example, a pattern of neurostimulation pulses to be delivered to the patient. Examples of such waveform segments include pulses, bursts (each burst including a group of the pulses), trains (each train including a group of the bursts), and sequences (each sequences including a group of the pulses), and the like.

The GUI allows for composition of one of more waveforms and waveform segments. The GUI comprises a library management area which allows the user to manage the waveforms and waveform segments stored in on a storage device. The GUI also comprises waveforms and waveform segment editors and each display a composition area for the on the display screen. The displayed composition area allows the user to compose each waveform and waveform segment. The composition includes editing a waveform or waveform segment selected from the waveforms or waveform segments stored in external storage device or retrieved from the library. The composition also includes creating new waveforms or waveform building blocks to be added to the waveforms and waveform building blocks that are stored in a storage device or library. These waveform segments or building blocks can be defined in terms of a number of repetitions of some base unit, or a duration (e.g., elapsed or relative time). Segments may also be nested and looped.

In various embodiments, each type of the plurality of types of the waveform segments represents a level of a plurality of levels of the waveform. A program building block of each level may include one or more waveforms and waveform segments of one or more lower levels. In various embodiments, the plurality of types of the waveform segments may include, but is not limited to, pulses (e.g., each having a time scale of milliseconds), bursts (e.g., each having a time scale of milliseconds to seconds) each including a group of the pulses, trains (e.g., each having a time scale of milliseconds to minutes) each including a group of the bursts, and sequences (e.g., each having a time scale of minutes to weeks) each including a group of the pulses, bursts, and trains. Pauses (each being a time interval during which no pulse is to be delivered) may each be placed between two of the waveform segments. The sequences may each be composed for a specific therapy. In some examples, the hierarchy in the order from the lowest to the highest is: pulse, burst, train, and sequence. By utilizing the waveform segments, the GUI allows for the definition of complex programs that cover long temporal periods and contain many different pulses, bursts, trains, and sequences. For example, the waveform may contain one or more different pulses, the one or more pulses differing from each other by zero or more pulse characteristics. The waveform may contain one or more different bursts, the one or more bursts differing from each other by zero or more burst characteristics. The waveform may contain one or more different trains, the one or more different trains differing from each other by zero or more burst characteristics.

Returning to FIG. 13, at operation 1320 when the user is satisfied with the created program, the composer builds the composition file. The composition file comprises information necessary to deliver the neurostimulation therapy described by the created waveform or waveform segment. The composition file may also comprise metadata associated with the created waveform or waveform component. Example metadata may include statistical information on the neurotherapy such as duration, average pulse width, maximum pulse width, minimum pulse width, average amplitude, maximum amplitude, minimum amplitude, and the like. Other example metadata may include information about who created the program, when it was created, and the like.

At operation 1330 the composition may be stored. For example, on a local storage device of the composer, or by sending it to a composition library. At operation 1340 the composition may be sent to a programmer. This may be the result of the programmer requesting the composition from the composer (e.g., as a result of the user of the programmer browsing and selecting this composition from all compositions in storage of the programmer) or may be the result of a user on the composer explicitly sending (e.g., pushing) the composition to the programmer. In some examples, where the composer and the programmer are resident on the same device, sending the composition to the programmer may be part of storing the composition at operation 1330. In other examples, it may include sending an interprocess communication to the programmer. In still other examples, where both the composer and the programmer are provided by the same application, sending the composition to the programmer may include populating one or more data structures that are used by the programmer.

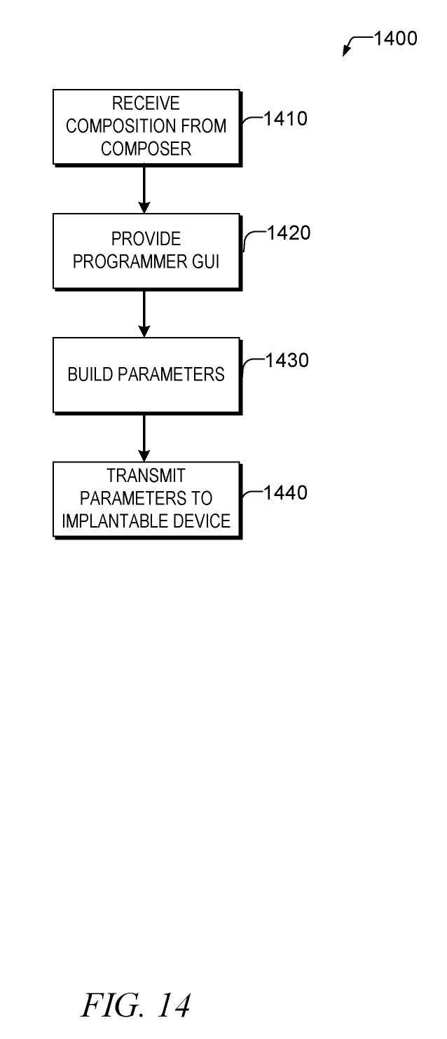

Turning now to FIG. 14, a flowchart of a method of a programmer 1400 is shown according to some examples of the present disclosure. At operation 1410 the programmer may receive the composition from the composer. At operation 1420 the programmer may provide a user interface, such as a Graphical User Interface (GUI) to a user to allow the user to view, select, and edit waveforms and waveform segments. As previously discussed, in some examples, certain aspects of the waveforms or waveform segments may not be editable by the GUI on the programmer device. Once a user is ready to transfer the program to a connected implantable device, the programmer may build a set of one or more stimulation parameters to send to the implantable device. In some examples, this may simply be the composition. In some examples, the composition may be stripped of non-essential matter (e.g., metadata) that is not necessary for providing the stimulation therapy to accommodate the implantable device's limited memory. In other examples, the composition (with any changes made by the programmer GUI) may be translated into a set of one or more stimulation parameters. At operation 1440 the stimulation parameters may be transmitted to the implantable device.

In some examples, the programming device may test the waveform prior to activating the program on the implantable device. As some of the waveforms generated involve sequences of stimulation which run for long periods of time—longer than those typically experienced in a clinical programming visit—it is not possible to fully test the entire waveform during that time. As a result, the programmer may take steps to ensure patient safety and program tolerability. In one example, all waveforms programmed into the device must have been run before they can be activated. Each waveform may have a “has run” bit which is flipped after the waveform was run—e.g., for a set time limit.

In some examples, to ensure that the waveform or waveform segment was adequately tested for the set time limit, the programming device may choose one or more portions of the waveform or waveform segment to test. For example, the programming device may automatically select using the programming application portions of the program that match one or more criteria. Criteria may be, for example, a pulse width, amplitude, frequency, or total energy delivered of the stimulation present on a given area of one or more leads for at least some electrode configurations that meet predetermined thresholds. The programming device may consider various combinations of parameter configurations. For example, a pulse width that exceeds a predetermined threshold with a low amplitude that is under a predetermined threshold.

The programming device converts the waveform into a test waveform that adequately simulates these criteria. One example conversion is simply finding the sections of the waveform exhibiting these characteristics and cropping the program to only contain these sections. The waveform is then programmed onto the stimulation device and may be run in a clinical setting to monitor the patient. In some examples, a clinician is required to confirm that a waveform has been adequately evaluated (e.g., by an acknowledgement on the UI) before the waveform may be accessed by the patient. If the patient responds favorably, the clinician may flip the “has run” bit and transfer the whole waveform to the implantable neurostimulation device.

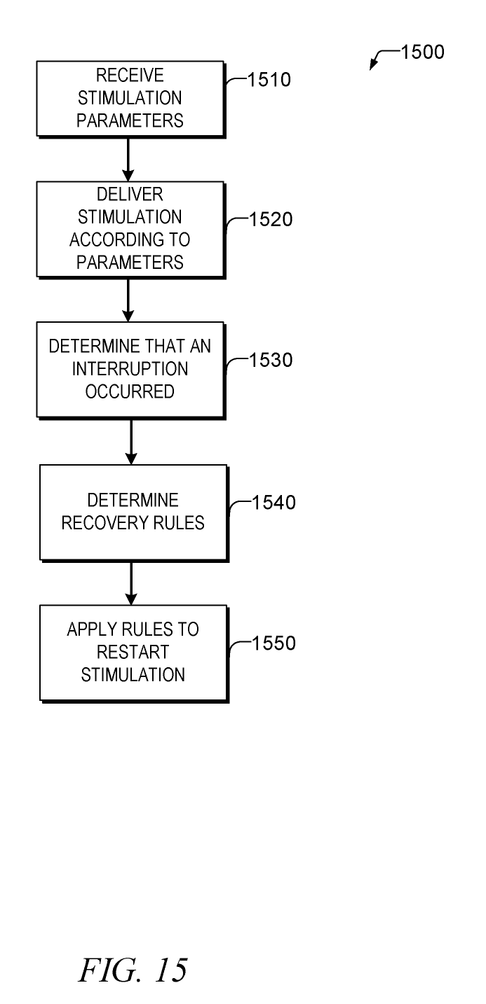

Turning now to FIG. 15, a flowchart of a method 1500 run by an implantable medical device is shown according to some examples of the present disclosure. At operation 1510 the implantable medical device receives the stimulation parameters. At operation 1520, the implantable medical device delivers stimulation according to the parameters. In some examples, various actions may be taken to initiate the therapy of the program. Actions may include selection of the program on a remote of the user, receipt of a stimulation start signal from the programmer, satisfaction of one or more predicate conditions (e.g., time, date, sensor reading, or other conditions) and the like. In some examples, the therapy continues until it ends or is stopped by the user.

While the therapy may complete as desired, various events may result in disruption of the stimulation. For example, the device may stop stimulation for health and safety reasons in response to detection of an internal or external event detected by one or more sensors (e.g., a strong magnetic field, electric transient, physiological sensor reading indicating that the therapy should be stopped, or the like), as a result of a software fault, a signal from the user to stop the stimulation, or the like. This interruption is detected at operation 1530. Once the device recovers from this situation (e.g., the software reboots, the sensors indicates it is safe to proceed, or the like), at operation 1540 the recovery rules are determined. For example, the recovery rules may be programmed into the device, stored in storage on the device (either at manufacturing or during programming), as part of the program, or the like. At operation 1550 the recovery rules determined in operation 1540 are applied to restart the stimulation. For example, the implantable medical device may determine how long stimulation was interrupted and may utilize that in determining how to proceed. For example, a pulse train may resume at the point where it stopped, at a point where it would have been had stimulation not been stopped, or start over. In some examples, the interruption may be saved in a log in a storage device on the implantable medical device and reported to a programming device or a remote of the user.

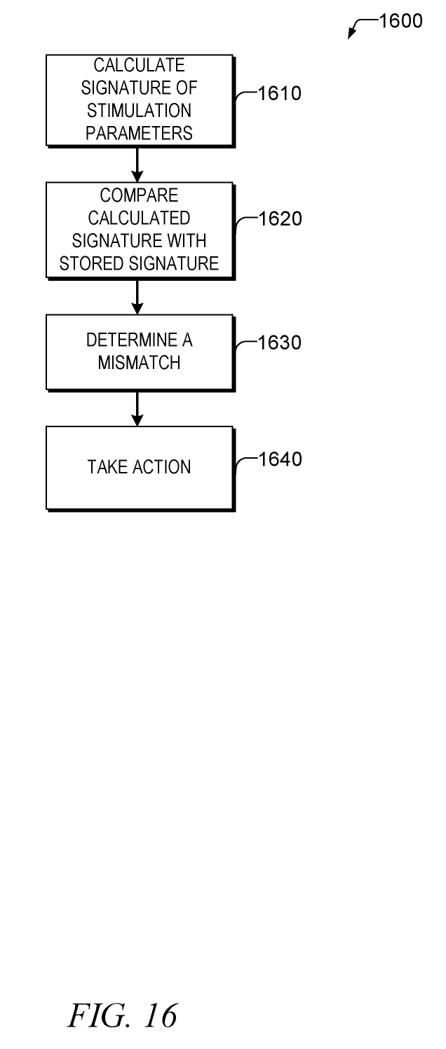

FIG. 16 shows a flowchart of a method 1600 of an implantable medical device according to some examples of the present disclosure. Method 1600 may be run periodically (on the stimulation parameters in a volatile memory of the implantable medical device), when the stimulation parameters are loaded prior to beginning therapy, on power-on of the device, or the like. Method 1600 and method 1500 may be run concurrently, in parallel, in succession, and the like. For example, Method 1600 may be run prior to operation 1520, and periodically during 1520 to ensure that the parameters are still valid. At operation 1610, the signature of the stimulation parameters (e.g., the composition) may be determined. The signature may be a hash value, a digital signature, or the like and may be calculated using one or more algorithms, such as an MD-5 hash algorithm. At operation 1620 the calculated signature may be compared with the signature stored for this program to determine if it matches or does not match. At operation 1630 if the implantable medical device determines that the signature does not match, then at operation 1640 a predetermined action is taken. The action may be programmed into the implantable medical device, indicated in the composition, or programmed by the programming device. In a first example action, the implantable device loads a duplicate copy of the stimulation parameters. In a second example action, a backup waveform is loaded. The backup waveform may be a different program than the program for which the signature did not match the stored signature. The backup waveform may be computed by the composer, the programmer, the implantable medical device, or may be generated by the user (e.g., through the remote device). The signature mismatch may be logged and the user and/or clinician are notified.

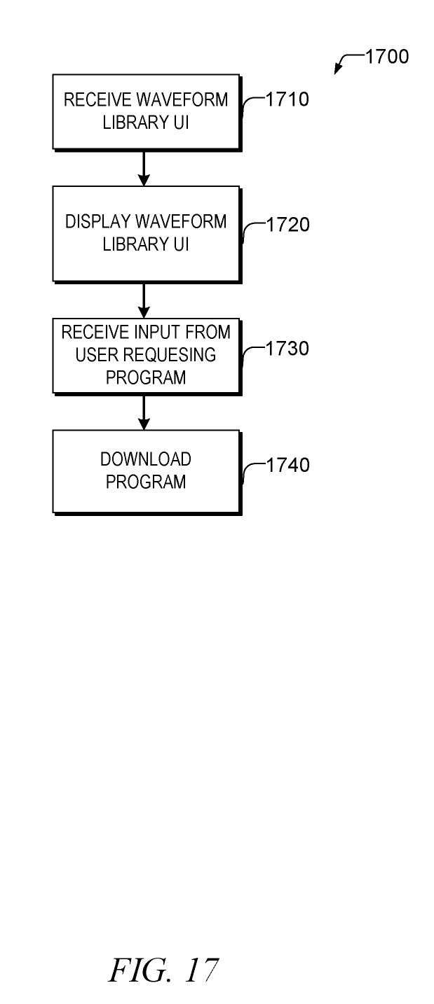

FIG. 17 shows a flowchart of a method 1700 of a programmer or composer device accessing a waveform library according to some examples of the present disclosure. At operation 1710 the device contacts the waveform library and requests the waveform library UI (e.g., GUI). For example, the device contacts the waveform library over a network (e.g., a WAN, LAN, or other network) and requests a user interface descriptor such as an HTML or other web-based application. The device then displays this UI at operation 1720. As part of this UI, information on a plurality of waveforms or waveform segments may be displayed. In some example UIs, a user may browse, search for, see details on, and select one or more waveforms and waveform segments. The UI may comprise one or more individual pages, which may be separately requested from the waveform library as requested by the user. For example, the waveform library may be an online Internet site. At operation 1730, the user may request to download a waveform or waveform segment, and the device may receive this request as an input. At operation 1740, the device may download a waveform or waveform segment from the waveform library. The program file may then be registered with or otherwise made available to the composer or configuration applications running on the device.

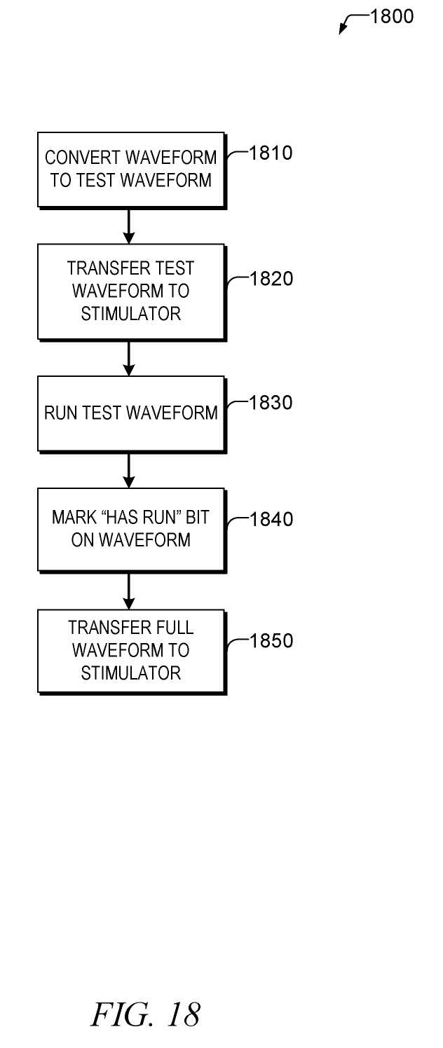

FIG. 18 shows a flowchart of a method 1800 of testing a program prior to deployment on the implantable medical device. At operation 1810 the configuration application converts the waveform to a test waveform. This may be done in response to a request from the clinician. The configuration application may search through the waveform to find waveform segments that match one or more predetermined criteria. Example criteria include minimum or maximum pulse width, amplitude, frequency, and the like. These waveform segments may then be transferred to the stimulator at operation 1820. All segments may be concatenated together in a single waveform in some examples, but in other examples, each segment may be loaded and tested (operations 1820-1830) sequentially. At operation 1830 the test waveform is run to gauge patient reactions and tolerability. In some examples, sensors in the implantable device may deliver information on tolerability, and may be used by the clinician to assess tolerability. At operation 1840, if desired, the “has run” bit may be set in the program to indicate it is safe to run. At operation 1850 the full program may be transferred to the stimulator if desired by the clinician.

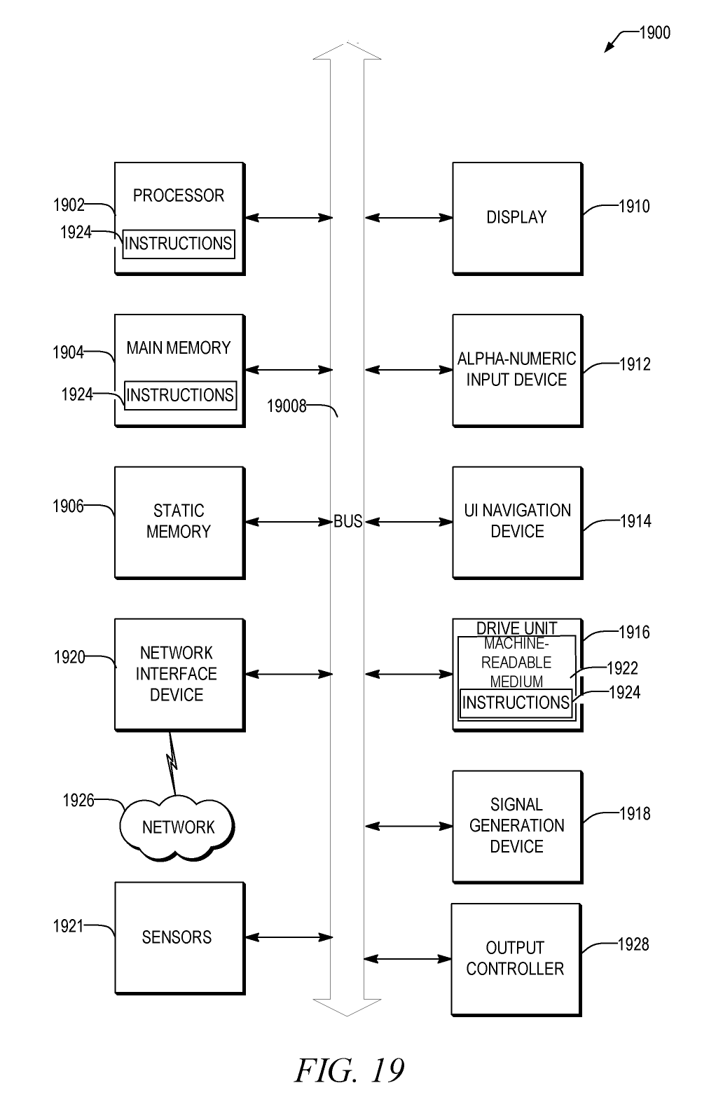

FIG. 19 illustrates a block diagram of an example machine 1900 upon which any one or more of the techniques (e.g., methodologies) discussed herein may perform. In alternative embodiments, the machine 1900 may operate as a standalone device or may be connected (e.g., networked) to other machines. In a networked deployment, the machine 1900 may operate in the capacity of a server machine, a client machine, or both in server-client network environments. In an example, the machine 1900 may act as a peer machine in peer-to-peer (P2P) (or other distributed) network environment. The machine 1900 may be a composer device, a programmer device, an implantable medical device, personal computer (PC), a tablet PC, a set-top box (STB), a personal digital assistant (PDA), a mobile telephone, a smart phone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 1900 may include a hardware processor 1902 (e.g., a central processing unit (CPU), a graphics processing unit (GPU), a hardware processor core, or any combination thereof), a main memory 1904 and a static memory 1906, some or all of which may communicate with each other via an interlink (e.g., bus) 1908. The machine 1900 may further include a display unit 1910, an alphanumeric input device 1912 (e.g., a keyboard), and a user interface (UI) navigation device 1914 (e.g., a mouse). In an example, the display unit 1910, input device 1912 and UI navigation device 1914 may be a touch screen display. The machine 1900 may additionally include a storage device (e.g., drive unit) 1916, a signal generation device 1918 (e.g., a speaker), a network interface device 1920, and one or more sensors 1921, such as a global positioning system (GPS) sensor, compass, accelerometer, or other sensor. The machine 1900 may include an output controller 1928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1916 may include a machine readable medium 1922 on which is stored one or more sets of data structures or instructions 1924 (e.g., software) embodying or utilized by any one or more of the techniques or functions described herein. The instructions 1924 may also reside, completely or at least partially, within the main memory 1904, within static memory 1906, or within the hardware processor 1902 during execution thereof by the machine 1900. In an example, one or any combination of the hardware processor 1902, the main memory 1904, the static memory 1906, or the storage device 1916 may constitute machine readable media.

While the machine readable medium 1922 is illustrated as a single medium, the term “machine readable medium” may include a single medium or multiple media (e.g., a centralized or distributed database, and/or associated caches and servers) configured to store the one or more instructions 1924.

The term “machine readable medium” may include any medium that is capable of storing, encoding, or carrying instructions for execution by the machine 1900 and that cause the machine 1900 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine readable media may include non-transitory machine readable media. In some examples, machine readable media may include machine readable media that is not a transitory propagating signal.

The instructions 1924 may further be transmitted or received over a communications network 1926 using a transmission medium via the network interface device 1920. The Machine 1900 may communicate with one or more other machines utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 1920 may include one or more physical jacks (e.g., Ethernet, coaxial, or phone jacks) or one or more antennas to connect to the communications network 1926. In an example, the network interface device 1920 may include a plurality of antennas to wirelessly communicate using at least one of single-input multiple-output (SIMO), multiple-input multiple-output (MIMO), or multiple-input single-output (MISO) techniques. In some examples, the network interface device 1920 may wirelessly communicate using Multiple User MIMO techniques.

What is claimed is:

1. A method comprising:
using one or more computer processors:
providing a composer graphical user interface (GUI), the composer GUI providing a waveform editor, the waveform editor permitting a user to create a waveform for providing neurostimulation therapy;
building a composition comprising information on the waveform, the composition in a first format; and
sending the composition to a programmer, the programmer programming an implantable medical device with stimulation parameters in a second format different than the first format, based on the composition.

2. The method of claim 1, wherein the waveform comprises at least one sequence comprising one or more trains, the one or more trains comprising one or more bursts, the one or more bursts comprising one or more pulses.

3. The method of claim 1, wherein the programmer is executing on the one or more computer processors, and wherein sending the composition to the programmer comprises one or more of: sending the composition using interprocess communication, or storing the composition in a data storage device in a location accessible by the programmer.

4. The method of claim 1, wherein the programmer is executing on a separate programming device and wherein sending the composition to the programmer comprises sending the composition over a network.

5. The method of claim 1, wherein a first parameter of the waveform is marked in the composition as read only.

6. The method of claim 1, wherein a first portion of the composition is locked to prevent a first parameter of the waveform to prevent modification by the programmer.

7. The method of claim 1, comprising uploading the composition to a waveform library.

8. The method of claim 1, wherein the composer GUI allows the user to create a waveform segment family comprising a plurality of waveform segments in the waveform matching criteria defined by the user, and wherein the method further comprises changing a characteristic of all waveform segments in the pulse family responsive to a user input.

9. A system comprising:
a composer device comprising:
a processor;
a memory communicatively coupled to the processor and comprising instructions, which when executed by the processor, cause the processor to perform operations comprising:
providing a composer graphical user interface (GUI), the composer GUI providing a waveform editor, the waveform editor permitting a user to create a waveform for providing neurostimulation therapy;
building a composition comprising information on the waveform, the composition in a first format; and
sending the composition to a programmer, the programmer programming an implantable medical device with stimulation parameters in a second format different than the first format, based on the composition.

10. The system of claim 9 comprising:
a programmer device comprising:
a second processor;
a second memory communicatively coupled to the second processor and comprising instructions, which when executed by the processor, cause the processor to perform operations comprising:
receiving the composition;
connecting to the implantable medical device;
translating the waveform of the composition into a plurality of stimulation parameters; and
uploading the plurality of stimulation parameters to the implantable medical device.

11. The system of claim 10 wherein the programmer operations comprise:
providing a programmer GUI, the programmer GUI displaying information on the waveform;
determining that a first parameter of the waveform is marked in the composition as read only; and in response, preventing editing of the first parameter by a user of the programmer device.

12. The system of claim 11, wherein the programmer operations comprise:
connecting to the implantable medical device;
creating a test waveform from the waveform, the test waveform a waveform that is shorter in length and comprising at least one waveform segment with a maximum stimulation of the waveform.

13. The system of claim 9, wherein the programmer is executing on the processor, and wherein the operations of sending the composition to the programmer comprises one or more of: sending the composition using interprocess communication, or storing the composition in a data storage device in a location accessible by the programmer.

14. The system of claim 9, wherein a first portion of the composition is marked in the composition as read only.

15. The system of claim 9, wherein a first portion of the composition is locked to prevent a first parameter of the waveform from being modified by the programmer.

16. The system of claim 9, comprising uploading the composition to a waveform library.

17. The system of claim 9, wherein the composer GUI allows the user to create a waveform segment family comprising a plurality of waveform segments in the waveform matching criteria defined by the user, and wherein the operations further comprises changing a characteristic of all waveform segments in the waveform segment family responsive to a user input.

18. A machine-readable medium, comprising instructions, which when performed by a machine, causes the machine to perform operations comprising:
providing a composer graphical user interface (GUI), the composer GUI providing a waveform editor, the waveform editor permitting a user to create a waveform for providing neurostimulation therapy;
building a composition comprising information on the waveform, the composition in a first format; and
sending the composition to a programmer, the programmer programming an implantable medical device with stimulation parameters in a second format different than the first format, based on the composition.

19. The machine-readable medium of claim 18, wherein the waveform comprises one or more sequences comprising one or more trains, the one or more trains comprising one or more bursts, the one or more bursts comprising one or more pulses.

20. The machine-readable medium of claim 18, wherein the operations comprise running the programmer, and wherein the operations of sending the composition to the programmer comprises one or more of: sending the composition using interprocess communication, or storing the composition in a data storage device in a location accessible by the programmer.
